(19) United States
(12) Patent Application Publication
(10) Pub. No.: US 2019/0030205 A1
(43) Pub. Date: Jan. 31, 2019

(54) WOUND DRESSINGS AND METHODS

(71) Applicant: O.R. Science Inc., Edmonton (CA)

(72) Inventors: Todd MCMULLEN, Edmonton (CA); Robert BURRELL, Sherwood Park (CA)

(21) Appl. No.: 16/074,871

(22) PCT Filed: Feb. 3, 2017
(86) PCT No.: PCT/CA2017/050125

§ 371 Date: Aug. 2, 2018

Related U.S. Application Data
(60) Provisional application No. 62/291,254, filed on Feb. 4, 2016.

(51) Int. Cl. A61L 15/44; A61F 13/00; A61F 13/02

(52) U.S. Cl. CPC A61L 15/44; A61F 13/00063; A61L 15/46; A61F 2013/0028; A61F 13/0203

(57) ABSTRACT

A wound dressing, a method for treating a wound subject, a use of a wound dressing, and a wound dressing kit. The wound dressing comprises a pressure dressing, a moisture control core, and a substrate comprising an antimicrobial agent, the substrate in fluid communication with at least a portion of the moisture control core.

### CROSS REFERENCE TO RELATED APPLICATIONS

This application claims the benefit of priority of U.S. Provisional Patent Application No. 62/291,254, filed Feb. 4, 2016, the entire contents of which is hereby incorporated by reference.

### FIELD

The present disclosure relates generally to a wound dressings and methods.

### BACKGROUND

Wound care, including acute wounds such as surgical and traumatic wounds, as well as chronic wounds, is a multibillion dollar industry. Physicians and surgeons primarily use wound dressings to prevent infection but are too often left to deal with cosmetic and functional flaws that may be extensive such as hypertrophic scarring. Surgical scars remain one the most important focal points for patient concerns following surgery. A number of creams, ointments and other treatments at dermatological offices focus on treating scars after forming.

The lack of scar control, in most cases, is related to the process of scar formation.

The need for an effective dressing that reduces scarring and improves wound healing is well established in the medical community.

### SUMMARY

It is an object of the present disclosure to obviate or mitigate at least one disadvantage of previous.

In one aspect, there is provided a wound dressing comprising: a pressure dressing, a moisture control core, and a substrate comprising an antimicrobial agent, the substrate in fluid communication with at least a portion said moisture control core.

In another aspect, there is provided a wound dressing comprising: a pressure dressing, and a moisture control core.

In another aspect, there is provided a wound dressing comprising: a pressure dressing and a substrate comprising an antimicrobial agent.

In one example, the wound dressing further comprising a substrate comprising an antimicrobial agent, the substrate in fluid communication with at least a portion said moisture control core.

In another aspect, there is provided a wound dressing comprising: a moisture control core, and a substrate comprising an antimicrobial agent, the substrate in fluid communication with at least a portion said moisture control core.

In one example, the wound dressing further comprising a pressure dressing.

In one example, said antimicrobial agent is a noble metal or metallic ion with antimicrobial properties.

In one example, said antimicrobial agent is Ag, Au, Pt, Pd, Ir, Cu, Sn, Sb, Bi, or Zn.

In one example, said noble metal is in a concentration of about 1 ppm to about 3025 ppm.

In one example, said noble metal is in a concentration of about 50 ppm to about 200 ppm.

In one example, said antimicrobial agent comprises silver nanoparticles, elemental silver, zero valent silver, multivalent silver ions carried by zirconium phosphate (ZP-Ag), silver containing compounds such as silver sulfadiazine, or related compounds.

In one example, said substrate further comprising a therapeutic agent.

In one example, said therapeutic agent is one or more of an antibiotic, an anti-viral agent, an anti-protozoal agent, an anti-parasitic agent, or an anti-inflammatory agent.

In one example, said antibiotic is a β-lactam antibiotics, macrolides, monobactams, rifamycins, tetracyclines, chloramphenicol, clindamycin, lincomycin, fusidic acid, novobiocin, fosfomycin, fusidate sodium, capreomycin, colistimethate, gramicidin, minocycline, doxycycline, bacitracin, erythromycin, nalidixic acid, vancomycin, or trimethoprim.

In one example, said p-lactam antibiotics is ampicillin, aziocillin, aztreonam, carbenicillin, cefoperazone, ceftriaxone, cephaloridine, cephalothin, cloxacillin, moxalactam, penicillin G, piperacillin, ticarcillin, or any combination thereof.

In one example, said anti-inflammatory agent is a steroidal anti-inflammatory or non-steroidal anti-inflammatory.

In one example, said substrate comprises Acticoat®.

In one example, said moisture control core comprises cotton, rayon, rayon/polyester, cellulose or cellulose derivatives.

In one example, said moisture control core comprises surgical gauze.

In one example, said moisture control core has a width of about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, about 20 mm, about 21 mm, about 22 mm, about 23 mm, about 24, or about 25 mm.

In one example, said pressure dressing comprises gauze, adhesive tape, bandages, steri-strips, or adhesive bandages and pads.

In one example, said pressure dressing comprises steri-strips, wherein said moisture control core comprises surgical gauze, and said substrate comprises Acticoat®.

In one example, said moisture control core is wetted with a fluid.

In one example, said fluid comprises water.

In another aspect there is provided a method of treating a wound subject, comprising: applying a wound dressing according to any one of claims 1 to 29 to the wound on the subject, wherein said wound dressing is maintained on the wound for a duration of at least about three days, and applied at a pressure of about 10 mmHg to about 100 mmHg.

In one example, said duration is about 3 to about 14 days, about 3 to about 21 days, about 3 to about 28 days, about 3 to about 35 days, about 3 to about 42 days, about 3 to about 49 days, about 3 to about 56 days, or greater than about 56 days, or more than about 3 days.

In one example, said pressure is about 10 mmHg, about 20 mmHg, about 30 mmHg, about 40 mmHg, about 50 mmHg, about 60 mmHg, about 70 mmHg, about 80 mmHg, about 90 mmHg, or about 100 mmHg.

In one example, said moisture control core maintains an environment with about 80% to about 100% relative humidity at the wound surface.

In another aspect there is provided a use of a wound dressing according to any one of claims 1 to 29, for treating a wound in a subject, said wound dressing is adapted for application to said wound on said subject for a duration of at least about three days, at a pressure of about 10 mmHg to about 100 mmHg.

In one example, said duration is about 3 to about 14 days, about 3 to about 21 days, about 3 to about 28 days, about 3 to about 35 days, about 3 to about 42 days, about 3 to about 49 days, about 3 to about 56 days, or greater than about 56 days, or more than about 3 days.

In one example, said pressure is about 10 mmHg, about 20 mmHg, about 30 mmHg, about 40 mmHg, about 50 mmHg, about 60 mmHg, about 70 mmHg, about 80 mmHg, about 90 mmHg, or about 100 mmHg.

In one example, said moisture control core maintains an environment with about 80% to about 100% relative humidity at the wound surface.

In another aspect there is provided a wound dressing kit, comprising: a pressure dressing, a moisture control core, and a substrate comprising an antimicrobial agent, the substrate adapted for fluid communication with at least a portion said moisture control core.

In another aspect there is provided a wound dressing kit comprising: a pressure dressing, and a moisture control core.

In one example, further comprising a substrate comprising an antimicrobial agent, the substrate in fluid communication with at least a portion said moisture control core.

In another aspect there is provided a wound dressing comprising: a moisture control core, and a substrate comprising an antimicrobial agent, the substrate in fluid communication with at least a portion said moisture control core.

In one example, further comprising a pressure dressing.

In one example, said antimicrobial agent is a noble metal or metallic ion with antimicrobial properties.

In one example, said antimicrobial agent is a noble metal or metallic ion with antimicrobial properties.

In one example, said antimicrobial agent is Ag, Au, Pt, Pd, Ir, Cu, Sn, Sb, Bi, or Zn.

In one example, said noble metal is in a concentration of about 1 ppm to about 3025 ppm.

In one example, said noble metal is in a concentration of about 50 ppm to about 200 ppm.

In one example, said antimicrobial agent comprises silver nanoparticles, elemental silver, zero valent silver, multivalent silver ions carried by zirconium phosphate (ZP-Ag), silver containing compounds such as silver sulfadiazine, or related compounds.

In one example, said substrate further comprising a therapeutic agent.

In one example, said therapeutic agent is one or more of an antibiotic, an anti-viral agent, an anti-protozoal agent, an anti-parasitic agent, or an anti-inflammatory agent.

In one example, said antibiotic is a β-lactam antibiotics, macrolides, monobactams, rifamycins, tetracyclines, chloramphenicol, clindamycin, lincomycin, fusidic acid, novobiocin, fosfomycin, fusidate sodium, capreomycin, colistimethate, gramicidin, minocycline, doxycycline, bacitracin, erythromycin, nalidixic acid, vancomycin, or trimethoprim.

In one example, said β-lactam antibiotics is ampicillin, aziocillin, aztreonam, carbenicillin, cefoperazone, ceftriaxone, cephaloridine, cephalothin, cloxacillin, moxalactam, penicillin G, piperacillin, ticarcillin, or any combination thereof.

In one example, said anti-inflammatory agent is a steroidal anti-inflammatory or non-steroidal anti-inflammatory.

In one example, substrate further comprising an anesthetic agent.

In one example, said substrate comprises Acticoat®.

In one example, said moisture control core comprises cotton, rayon, or rayon/polyester, cellulose, or cellulose derivatives.

In one example, said moisture control core comprises surgical gauze.

In one example, said moisture control core has a width of about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, about 20 mm, about 21 mm, about 22 mm, about 23 mm, about 24, or about 25 mm.

In one example, said pressure dressing comprises gauze, adhesive tape, bandages, steri-strips, or adhesive bandages and pads.

In one example, said pressure dressing comprises steri-strips, wherein said moisture control core comprises surgical gauze, and said substrate comprises Acticoat®.

In one example, further comprising a fluid.

In one example, said fluid comprises water.

A method as described herein.

A use as described herein.

A wound dressing as described herein.

A kit as described herein.

Other aspects and features of the present disclosure will become apparent to those ordinarily skilled in the art upon review of the following description of specific embodiments in conjunction with the accompanying figures.

### BRIEF DESCRIPTION OF THE DRAWINGS

Embodiments of the present disclosure will now be described, by way of example only, with reference to the attached Figures.

FIG. 1 depicts one embodiment of a wound dressing; and

FIG. 2 depicts a comparison of the dressing of the present application compared to a standard dressing, after two weeks of application to a wound (Panel A), or after eight weeks of application to a wound (Panel B).

### DETAILED DESCRIPTION

Generally, the present disclosure provides a wound dressing and method.

As described herein, there is provided a wound dressing for promoting and enhancing wound healing in a subject, also referred to as treating a wound in a subject.

It has been surprisingly determined that a combination of (i) an antimicrobial composition, that is (ii) applied under pressure to a wound, and (iii) under conditions which keep the wound moist, results in enhanced healing of the wound, as would be expected from either the antimicrobial composition alone or pressure applied to the wound alone.

In one aspect, the wound dressing applies a substrate comprising an antimicrobial agent to a wound in a subject, and applies pressure to the wound, under conditions which keep the wound moist.

In another aspect, the wound dressing applies a substrate comprising an antimicrobial agent and an anti-inflammatory agent to a wound in a subject, and applies pressure to the wound, under conditions which keep the wound moist. In one example, the antimicrobial agent also exhibits the properties of an anti-inflammatory agent.

A “subject” or “patient” as used herein, refers to any mammal or non-mammal that would benefit from treatment of a wound. In certain examples a subject or patient includes, but is not limited to, humans, farm animals (cows, sheep, pigs, and the like), companion animals (such as cats, dogs and horses, and the like), non-human primates, and rodents (such as mice and rats).

In a specific embodiment, the subject is a human, including human adults, children, and the elderly.

A “wound”, as used herein, refers to injuries to the skin and subcutaneous tissue, initiated in different ways, and with varying characteristics.

Wounds may be classified into one of four grades depending on the depth of the wound: i) Grade I: wounds limited to the epithelium; ii) Grade II: wounds extending into the dermis; iii) Grade III: wounds extending into the subcutaneous tissue; and iv) Grade IV (or full-thickness wounds): wounds wherein bones are exposed (e.g., a bony pressure point such as the greater trochanter or the sacrum).

The term “partial thickness wound” refers to wounds that encompass Grades I-III; examples of partial thickness wounds include burn wounds, pressure sores, venous stasis ulcers, and diabetic ulcers. The term “deep wound” is meant to include both Grade III and Grade IV wounds. The present invention contemplates treating all wound types, including deep wounds and chronic wounds.

The term “chronic wound” refers to a wound that has not healed within 30 days. Wounds may be from trauma. In some examples the wound is from surgery. Surgical scars and incisions are made intentionally, for example, in the case of wound debridement and drain sites as well as skin shavings for Moh's surgery/Moh's procedures.

Wounds may also be caused by burns, psoriasis, atopic dermatitis, pressure sores, and the like.

In some examples, for example where the wound is in the form of a chronic wound, it may be necessary to surgically resect the chronically inflamed tissue to form a surgical wound and then to treat the surgical wound with the wound dressing described herein.

Wounds may be treated on a wide range of locations of the subject. For example, the wound dressing may be used for wound sites of application such as the head, neck, chest, abdominal and extremities.

As noted above, the wound dressing described herein is for promoting and enhancing wound healing in a subject.

“Promote wound healing” and “enhance wound healing” and “treating a wound”, and the like, refer to either the induction of the formation of granulation tissue of wound granulation tissue in the wound bed and/or the induction of epithelialization (i.e., the generation of new cells in the epithelium; also referred to as re-epithelialization). Wound healing is conveniently measured by decreasing wound area. In one example, wound healing is measured using the POSAS Observer scale. In some examples, cell remodeling of a wound follows re-epithelization.

Thus, the treatment of the wound may involve remedying a condition or symptoms, preventing the establishment of a condition or disease, or otherwise preventing, hindering, retarding, or reversing the progression of a condition or disease or other undesirable symptoms in any way whatsoever. In one example the treatment of the wound may result in any one or more of the following actions, processes or outcomes, minimization of inflammation, minimization of fibrosis, deposition of collagen, differentiation of stem cells into functional dermal cells, reduction in scar formation, reversal of scarring or fibrotic lesions, angiogenesis, re-epithelialization, granulation tissue formation, and/or cell remodeling.

As noted above, in one example, wound healing is measured using the POSAS Observer scale. Alternate methods of measuring wound healing are known to the skilled worked. See, for example, Fearmonti, R. et al. (2010) “A review of Scar Scales and Scar Measuring Devices” Open Access Journal Of Plastic Surgery. Page 354-363, the contents of which are incorporated by reference in its entirety. Non limiting examples include, The Vancouver Scar Scale (VSS), Manchester Scar Scale (MSS), Visual Analog Scale (VAS), and the Stony Brook Scar Evaluation Scale (SBSES).

In a specific example, the wound dressing comprises a pressure dressing, a moisture control core, and a substrate comprising an antimicrobial agent, the substrate is associated, and in fluid communication with, at least a portion said moisture control core.

In another specific example, the wound dressing comprises a pressure dressing, a moisture control core, and a substrate comprising an antimicrobial agent and an anti-inflammatory agent, the substrate is associated, and in fluid communication with, at least a portion said moisture control core.

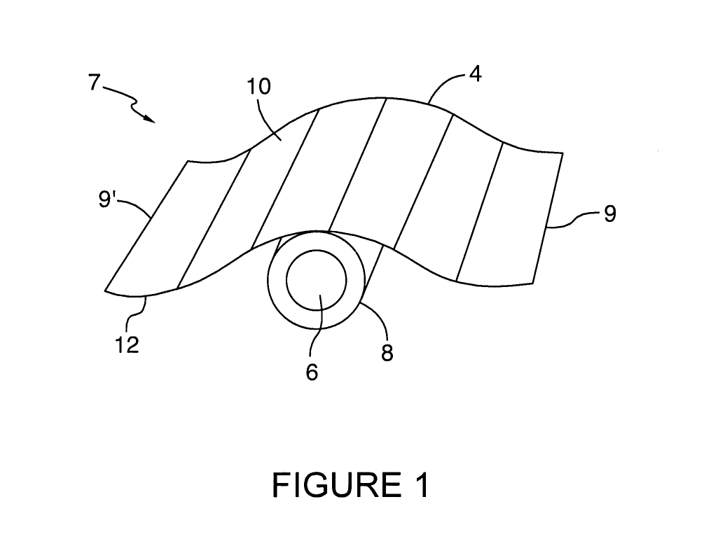

FIG. 1 depicts an example of wound dressing (2), comprising: pressure dressing (4), moisture control core (6), and a substrate (8) comprising an antimicrobial agent, said substrate (8) is associated with, and in fluid communication with, at least a portion of moisture control core (6). In some examples, the antimicrobial agent is also an anti-inflammatory agent. In some examples, the substrate (8) comprising an antimicrobial agent further comprises an anti-inflammatory agent.

The substrate is sized to be applied to and cover a wound in a subject. It will be appreciated that the size and shape of the substrate will vary according to the intended use, and, for example, the size and location of the wound to which it is applied.

In the Example of FIG. 1, substrate (8) comprising antimicrobial agent is generally depicted as surrounding the circumference of moisture control core (6). It will be appreciated that a substrate comprising an antimicrobial agent (8) may be sized and positioned to cover at least a portion of moisture control core (6).

In some examples, the substrate comprising an antimicrobial agent is integral with at least a portion of the outer surface of the moisture control core.

In some examples, the substrate comprising an antimicrobial agent is applied to at least a portion of the outer surface of the moisture control core. The substrate comprising an antimicrobial agent may be applied to the moisture control core fixedly or removably.

In some examples, the substrate comprising an antimicrobial agent is separate from the moisture control core. In such an example the antimicrobial containing substrate is applied to the wound, and the moisture control core is placed on the substrate comprising an antimicrobial agent.

In use, minimal moisture/liquid is lost from the wound dressing when applied to a subject.

In one example, the antimicrobial agent is nanocrystalline silver and the moisture/liquid form the moisture control core is sufficient to provide activation of the nanocrystalline silver for application to the wound, as well as aid in healing of the wound by preventing the drying scab overlay which will occur if the wound is allowed to dry out, which makes a worse scar in of itself.

In use, the moisture control core absorbs moisture or supplies moisture to keep the wound site at about 100% relative humidity. In another example, in use, the moisture core absorbs moisture or supplies moisture to keep the wound site at about 80% humidity to about 100% humidity. In some examples, the moisture control core absorbs moisture or supplies moisture to keep the wound site at about 100% relative humidity, about 95% humidity, about 90% humidity, about 85% humidity, or about 80% humidity.

The moisture creates an environment for wound healing while minimizing risk of infection. The moisture control core also acts as a pressure point so that pressure from the pressure dressing is transmitted through the moisture control core, to the wound.

In use, the pressure dressing is kept on the wound for about 3 to about 14 days. In some examples, the pressure dressing is kept on the wound for about 3 to about 21 days. In some examples, the pressure dressing is kept on the wound for about 3 to about 28 days. In some examples, the pressure dressing is kept on the wound for about 3 to about 35 days. In some examples, the pressure dressing is kept on the wound for about 3 to about 42 days. In some examples, the pressure dressing is kept on the wound for about 3 to about 49 days. In some examples, the pressure dressing is kept on the wound for about 3 to about 56 days. In some examples, the pressure dressing is kept on the wound for greater than about 56 days.

As noted above, the substrate comprising an antimicrobial agent is in fluid communication with the moisture control core. As discussed further below, the moisture control core, together with a vapor barrier, maintains an environment with about 80% to about 100% relative humidity at the wound surface. The substrate comprising an antimicrobial agent releases the antimicrobial agent(s) (e.g., atoms, ions, molecules or clusters of at least one of the antimicrobials contained within/on the substrate), into a fluid from within the moisture control core (e.g. water) and to the wound for an extended period of time.

In some examples, the extended period is about 3 to about 14 days. In some examples, the extended period is about 3 to about 21 days. In some examples, the extended period is about 3 to about 28 days. In some examples, the extended period is about 3 to about 35 days. In some examples, the extended period is about 3 to about 42 days. In some examples, the extended period is about 3 to about 49 days. In some examples, the extended period is about 3 to about 56 days. In some examples, the extended period is greater than about 56 days.

The moisture control core may be made from a variety of materials. In one example, the moisture control core is surgical gauze. In other examples, the moisture control core is cotton, rayon, rayon/polyester, cellulose, or cellulose derivatives, or combinations thereof. In some examples, the moisture control cores comprise Na CMC (sodium carboxymethyl cellulose). In some examples the moisture control core comprises AQUACEL® Ag dressing.

The moisture control core is sized and shape to provide the desired pressure to the wound. Thus, shape of the moisture control core will vary with the intended use and, for example, the size, shape and location of the wound to be treated. In the example of FIG. 1, moisture control core (8) is generally cylindrical. In other examples, moisture control core (8) is small to large flat sheets, or discs. In non-limiting examples, the moisture control core may be generally cylindrical, rectangular, spherical, elliptical, pyramidal, a rhombus, a parallelogram, a trapezoid, a trapezium, and the like.

In some examples, the moisture control core is sized to have a thickness of about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, about 20 mm, about 21 mm, about 22 mm, about 23 mm, about 24, or about 25 mm. The size of the moisture control core is sized, in part, to provide sufficient pressure to the wound.

In use, the fluid control core is wetted with a fluid. The fluid used to wet the moisture control core will vary with the intended use. In one example, water is the fluid used in the moisture control core.

In some examples, the moisture control core included additional compounds to promote fluid retention. In some examples, the moisture control core further included β-glucans.

The pressure dressing is configured for association with the moisture control core and removable attachment to a portion of a subject surrounding the wound. The pressure dressing is configured to apply a pressure to the wound though the moisture control core, thereby applying pressure to the antimicrobial containing substrate on the wound.

In the Example of FIG. 1, pressure dressing (4) is depicted as a being separate from moisture control core (6). It will be appreciated that pressure dressing (4) may be integral with moisture control core (6).

In some examples, the pressure dressing is configured to apply a pressure of about 10 mmHg to 100 mmHg to the wound. In some examples, the pressure dressing is configured to apply about 10 mmHg, about 20 mmHg, about 30 mmHg, about 40 mmHg, about 50 mmHg, about 60 mmHg, about 70 mmHg, about 80 mmHg, about 90 mmHg, about 100 mmHg, of pressure to the wound.

In one example, and as depicted in FIG. 1, pressure dressing (4) comprises a vapor barrier surface (10), and a second surface (12). The vapor barrier surface prevents or reduces passage of liquid across the pressure dressing (4) from the second surface (12), for example, from moisture with in moisture control core (6), or bodily fluids from the wound of the subject.

It will be appreciated that the vapor barrier prevents or limits the passage of both liquid and gas/vapor. In the dressing, the vapor barrier is intended to prevent or limit evaporation of, for example, water, as water vapor or the gas phase of water, from the surface of the wound. In this roll it also prevents or limits the loss of liquid water.

It will be appreciated that it will not, in all cases, be necessary to include vapor barrier surface (10). However, in this example, the wound dressing (2) maintains the desired moisture at the wound site while applied to the patient.

In FIG. 1, vapor barrier surface (10) is depicted as covering an entire surface of pressure dressing (4). It will be appreciated that vapor barrier may only cover a portion, or plurality of portions, or the surface of pressure dressing (4).

The vapor barrier surface (10) of pressure dressing (4), moisture control core (4), and antimicrobial containing substrate (8) are sized to cover the wound on the subject (or a desired portion of the wound on the subject), and maintain antimicrobial layer (8) moist.

In one example, the pressure dressing (4) is elastic tape that has indicia at an even spacing on its upward surface. As the elastomer is stretched the marks also stretch and the increase in space between the lines is directly proportional to the pressure generated on the wound surface. This facilitates applications of the desired pressure to the wound.

Examples of wound dressing/pressure dressing materials include but are not limited to gauze, adhesive tape, bandages, and commercially available wound dressings including but not limited to adhesive bandages and pads from the Band-Aid® line of wound dressings, adhesive bandages and pads from the Nexcare® line of wound dressings, adhesive bandages and non-adhesive pads from the Kendall Curity Tefla® line of wound dressings, adhesive bandages and pads from the Tegaderm® line of wound dressings, adhesive bandages and pads from the Steri-Strip® line of wound dressings, the COMFEEL® line of wound dressings, adhesive bandages and pads, the Duoderm® line of wound dressings, adhesive bandages and pads, the TEGADERM™ line of wound dressings, adhesive bandages and pads, the OPSITE® line of wound dressings, adhesive bandages and pads, the Mepore® line of wound dressings, adhesive bandages and pads.

In other examples, the pressure dressing is made from, for example, but not limited to, metal, latex, nylon, silicone, polyester, glass, ceramic, paper, cloth and other plastics and rubbers.

As will be appreciated, the moisture control core and antimicrobial containing substrate may be incorporated in an existing pressure dressing. In one example, in the case of a wound from thoracic surgery, the moisture control core and antimicrobial containing substrate may be used with a pressure dressing which is a chest binder or front-closing brassiere. In other examples, the pressure dressing is a post-surgical vest.

From the foregoing it will be appreciate that a variety of materials may be used for the pressure dressing. In some cases, the pressure dressing is elastomeric. In some cases, the pressure dressing is not elastomeric. The pressure dressing is selected based on the ability of to apply the suitable pressure to the wound.

As discussed above, the wound dressing comprises a substrate comprising an antimicrobial agent.

In one example, the antimicrobial agent comprises monocrystalline silver.

In a specific example, the antimicrobial agent comprises ACTICOAT®. In another example, ACTICOAT® if from the ACTICOAT® family of dressings.

Of the metallic ions with antimicrobial properties, silver is perhaps the best known due to its good bioactivity at low concentrations. This phenomena is termed oligodynamic action. In modern medical practice both inorganic and organic soluble salts of silver are used to prevent and treat microbial infections. While these compounds are effective as soluble salts, they do not provide prolonged protection due to loss through removal or complexation of the free silver ions. Typically, they must be reapplied at frequent intervals to overcome this problem. Reapplication is not always practical, especially where a dressing in a homecare situation is involved. The wound dressing described herein does not require reapplication of the antimicrobial agent.

In other example, the antimicrobial agents includes, but it not limited to, metallic ions, including but not limited to, Ag, Au, Pt, Pd, Ir (i.e. the noble metals), Cu, Sn, Sb, Bi and Zn.

In use, in the case of noble metals, the antimicrobial agent is used at a concentration of about 1 ppm to about 3025 ppm. In a specific example, the antimicrobial agent is used at a concentration of about 50 ppm to about 200 ppm.

In some examples, the substrate includes, but is not limited to nanostructured thin films as exemplified in U.S. Pat. No. 5,681,575, the contents of which are hereby incorporated by reference in their entirety. Therein, an antimicrobial containing substrate is described that provides a sustained release of an antimicrobial agent at therapeutically active levels.

In other examples, the antimicrobial used in in the antimicrobial containing substrates includes, but is not limited to, silver nanoparticles, elemental silver, zero valent silver, multivalent silver ions carried by zirconium phosphate (ZP-Ag), and silver containing compounds such as silver sulfadiazine and related compounds.

In some examples, the substrate comprising an antimicrobial agent may be produced using vacuum deposition techniques, on a suitable substrate, including but not limited to a polymer including high density polyethylene, nylon etc. as a thin polycrystalline film.

In some examples, the substrate includes, but is not limited to, flexible and/or elastomeric articles. The substrate may be films, sheets, tubes, fibers, and the like formed of polymeric materials that are either thermoplastic polymers or thermoset polymers. Exemplary polymeric substrates include, but are not limited to, thermoplastic and thermoset polymers that are optionally plactisized and include polyolefins, polyethylene, polypropylene, fluoropolymers, polyamides, polyethers, epoxies, polyvinyl chloride and plasticized polyvinylchloride, polyisoprene, polyisobutylene, block copolymers such as styrene-butadiene styrene and styrene-isoprene styrene, hydrogenated versions of these available from Kraton Polymers, metallocene polyolefins, rayon polyester, polyethylene terephthalate (PET), poly(meth)acrylates, polycarbonates, polystyrenes, polystyrene copolymers such as styrene acrylonitrile copolymers, polyesters, polyethersulfone, acrylics and acrylic copolymers, polyacrylamides, and polyurethanes, silicones such as two part cured polydiemthylsiloxanes, polymethyl methacrylates, natural rubber latex, polyisoprene, nitrile rubber, and the like, as well as combinations thereof including blends thereof and laminates thereof.

In some examples, the moisture control core and/or antimicrobial containing substrate further comprise one or more therapeutic agents for administration to the wound.

In some examples, the therapeutic agent is a small molecule and/or organic compound with pharmaceutical activity.

A therapeutic agent can be or comprise an antimicrobial agent and/or an anti-inflammatory agent.

A therapeutic agent may be an antibiotic. Examples of antibiotics include, but are not limited to, β-lactam antibiotics, macrolides, monobactams, rifamycins, tetracyclines, chloramphenicol, clindamycin, lincomycin, fusidic acid, novobiocin, fosfomycin, fusidate sodium, capreomycin, colistimethate, gramicidin, minocycline, doxycycline, bacitracin, erythromycin, nalidixic acid, vancomycin, and trimethoprim. For example, β-lactam antibiotics can be ampicillin, aziocillin, aztreonam, carbenicillin, cefoperazone, ceftriaxone, cephaloridine, cephalothin, cloxacillin, moxalactam, penicillin G, piperacillin, ticarcillin and any combination thereof. Other antimicrobial agents such as those described herein, may also be used in accordance with some embodiments of the present disclosure. For example, anti-viral agents, anti-protozoal agents, anti-parasitic agents, etc. may be of use.

Additionally or alternatively, a therapeutic agent may be an anti-inflammatory agent.

Examples of anti-inflammatory agents include, but are not limited to, steroidal and/or non-steroidal anti-inflammatory agents.

A therapeutic agent may be a mixture of pharmaceutically active agents. For example, a local anesthetic may be delivered in combination with an anti-inflammatory agent such as a steroid. Local anesthetics may also be administered with vasoactive agents such as epinephrine.

In use, in some examples, the pressure dressing is wrapped around a portion of the subject which has the wound to apply the pressure.

In some examples, the dressing includes an adhesive portion, which is compatible with the skin of the subject, and is used to adhere to a position of the subject proximal to the wound. By adhering, for example free ends (9, 9′) of the pressure dressing to areas of the subject, suitable pressure may be applied to moisture control core and in turn the wound.

In use, a pressure dressing as described herein is applied with a pressure of about 10 mmHg to 100 mmHg to the wound. In some examples, the pressure dressing is configured to apply about 10 mmHg, about 20 mmHg, about 30 mmHg, about 40 mmHg, about 50 mmHg, about 60 mmHg, about 70 mmHg, about 80 mmHg, about 90 mmHg, about 100 mmHg, of pressure to the wound.

In use, the pressure dressing is kept on the wound for about 3 to about 14 days. In some examples, the pressure dressing is kept on the wound for about 3 to about 21 days. In some examples, the pressure dressing is kept on the wound for about 3 to about 28 days. In some examples, the pressure dressing is kept on the wound for about 3 to about 35 days. In some examples, the pressure dressing is kept on the wound for about 3 to about 42 days. In some examples, the pressure dressing is kept on the wound for about 3 to about 49 days. In some examples, the pressure dressing is kept on the wound for about 3 to about 56 days. In some examples, the pressure dressing is kept on the wound for greater than about 56 days. In some example, the pressure dressing is kept on the wound for greater than about 3 days.

In a further embodiment there is provided a method of treating a wound including the step of applying a wound dressing described herein to a wound.

In a further embodiment there is provided a use of a wound dressing described herein for the treatment of a wound.

In another embodiment, there provided a wound dressing comprising a pressure dressing, and a moisture control core. The pressure dressing and moisture control core as described herein.

In another embodiment, there is provided a wound dressing comprising: a moisture control core, and a substrate comprising an antimicrobial agent, the substrate in fluid communication with at least a portion said moisture control core. The moisture control core and substrate comprising an antimicrobial agent as described herein.

In a further embodiment there is provided a kit including one of more of a wound dressing as described herein. In some embodiments, the kit includes the pressure dressing, the antimicrobial layer, and/or moisture control core.

In a further embodiment, there is provided a method of making a wound dressing as described herein.

Methods of the invention are conveniently practiced by providing the compounds and/or compositions used in such method in the form of a kit. Such a kit preferably contains the composition. Such a kit preferably contains instructions for the use thereof.

To gain a better understanding of the invention described herein, the following examples are set forth. It should be understood that these example are for illustrative purposes only. Therefore, they should not limit the scope of this invention in any way.

### EXAMPLES

### Example 1

### Anti-Inflammatory/Antimicrobial Dressing, Without Moisture

In the initial evaluations, the simple use of the anti-inflammatory/antimicrobial dressing elements in wound healing were not sufficient (without the addition of moisture) as there appeared to be inadequate activation and the changes in the scars were not as satisfactory. These evaluation were typically done in the initial stages simply using a simplified scar scoring platform, and typically involved between five and 15 patients for each iteration.

### Example 2

### Anti-Inflammatory/Antimicrobial Dressing, with Moisture, with Pressure

In this example, the wound dressing was used on neck incisions following surgery to remove the thyroid or parathyroid glands.

In all case the surgical rings were created with a scalpel under sterile conditions. All of the skin was cleaned with Solu-prep prior to the incision being made. The wounds were open for as short as 15 minutes or as long as three or four hours before being closed.

At the time the wound was ready to be closed, the skin areas cleansed with saline to make sure there is no dried blood and the wound edges are re-approximated in the deeper layers typically with sutures in the epidermis or dermal layer to bring the skin edges together. The suture material was typically a 5-0 vicryl.

### Standard Dressing

A standard dressing was the use of Steri-Strips directly to the incision.

Initially, a dressing was simply applied with the Acticoat directly underneath the steri-strips which are made by 3M. However the Steri strips often came off, or patients would call within 2 to 4 days indicating it was curling up at the edges or part of the Acticoat part of the dressing was sticking out underneath.

It was found with standard dressings that keeping the dressing in place required more work as the patients were unable to keep it on and the wounds were also drying out and this was clearly evident on the initial assessments of these patients at the two-week time point.

This resulted in the problem of poor skin contact as well as the wound drag out which causes granulation tissue making the scar worse.

Thus, the standard dressing was found to be unsatisfactory.

### Wound Dressing of the Present Invention

A wound dressing of the present application was developed to avoid or minimize the problems associated with standards dressings.

In one example, Acticoat was cut in a strip that were applied to the top of the incision with the inside skin at the middle with approximately a 1 cm margin on all sides both above and below the scar as well as to the left and right. Gauze (a moisture control core) was wetted with water such that gauze was moist. If the moisture control core was too wet, there is actually staining of the skin from the Acticoat which can be distressing for the patients although ultimately it is not harmful the discolouration can occur over the entire area of the Acticoat where it coats the skin if there's too much moisture in the form of free water.

Saline was not used as it may form AgCl crystals, which are not of assistance.

The application of pressure, together with the moisture, provided fixation of the wound. Fixation refers to minimizing movement of the wound, which is desirable for wound healing.

In one example, Steri-Strips were used to affix the underlying moist gauze which the Acticoat is then applied directly to, which is then applied to the wound. The Steri-Strips were placed over top of the dressing, typically with tincture of benzoin and surrounding skin to make sure it stays on at least two weeks.

The amount of gauze used to form the role was approximately 1 cm in diameter and was generally the same length as the incision essentially.

When the gauze was squeezed, substantially no additional water should came out in the form of water drops.

Mepore dressing may be used on top of the Steri-Strip, which was found to be helpful for those patients who have lots of movement.

During the evaluation, patients were informed that they need to try to keep the wound clean and not to soak it underneath water in terms of no swimming for at least two weeks and if they do shower they try to keep the dressing intact and patted it dry at the end of the shower.

It was found that, in the cases where the dressing had fallen off (early) or dried out, the wound dressing of the present invention was superior, based on the erythema and granulation scar forming over incision sites that had not had proper application of the dressing.

### Example 3

An embodiment of the present was dressing was made and used as follows. The Dressing was made by hand, using standard surgical gauze that was wetted with water, with a rolled up piece of gauze to provide the pressure under which a hand cut piece of the nanocrystalline silver was applied in a sandwich fashion with nanocrystalline silver strip on the bottom then the wetted small strip of gauze with the larger piece of gauze over top and finally at the very top of a Mepore® dressing which is a tough dressing on top which fixes it to the skin.

The wound dressing was used on neck incisions following surgery to remove the thyroid or parathyroid glands.

In all case the surgical rings were created with a scalpel under sterile conditions. All of the skin was cleaned with Solu-prep prior to the incision being made. The wounds were open for as short as 15 minutes or as long as three or four hours before being closed.

At the time the wound was ready to be closed, the skin areas cleansed with saline to make sure there is no dried blood and the wound edges are re-approximated in the deeper layers typically with sutures in the epidermis or dermal layer to bring the skin edges together. The suture material was typically a 5-0 vicryl.

Patients were informed that they need to try to keep the wound clean and not to soak it underneath water in terms of no swimming for at least two weeks and if they do shower they try to keep the dressing intact and patted it dry at the end of the shower.

All patients were seen at two weeks and then again at eight weeks to evaluate their scars and then in some cases again at three months later. Scoring of the wounds was carried out using the “POSAS Observer scale”.

In some experiments (not shown) in which pressure was not applied, the suboptimal results in terms of the scar and the integrity of the dressing itself was observed.

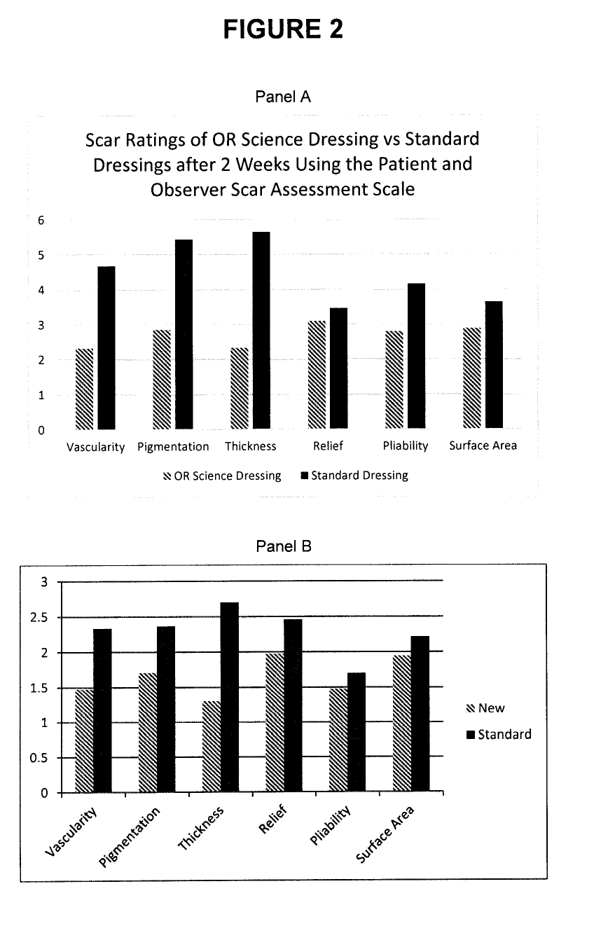

In FIG. 2, “Standard Dressing” refers to Steri-Strips applied directly to the wound. “OR Science Dressing” and “New” are one and the same, and refer to the dressing of the present invention.

FIG. 2, panel A, shows a comparison of the dressing of the present application, as described above, compared to a standard dressing, assessed using the POSAS Observer scale, two weeks following application of the dressing.

FIG. 2, panel B, shows a comparison of the dressing of the present application, as described above, compared to a standard dressing, assessed using the POSAS Observer scale, eight weeks following application of the dressing.

While not wishing to be bound by theory, it is believed the superiority of dressing of the present application, in which pressure is applied, is due to (i) the application of the pressure and the provision of stability by the dressing to minimize movement which mechanically reduces scar formation and (ii) the ability of the dressing to stay close to the incision for maximum hydration and the activity of the antimicrobial component, e.g., the nanocrystalline silver strip (Acticoat).

In use, the dressing of the present application may be applied to a wide range of surgical wounds, of varying size, and on a wide range of sites of the body.

Preferably, the dressing is on the subject about two weeks. In some examples, the dressing is on the subject for about 3 weeks. In some examples, the dressing is on the subject about 4 weeks. In some examples, the dressing is on the subject about 5 weeks. In some examples, the dressing is on the subject about 6 weeks. In some examples, the dressing is on the subject about 7 weeks. In some examples, the dressing is on the subject about 8 weeks. In some examples, the dressing is on the subject more than about 8 weeks.

For example, in patients that have been observed in the range of 2 to 8 weeks, have better results especially in terms of erythema and minimizing raised edges.

While not wishing to be bound by theory, it is thought that the longer applications of the dressing permit re-epithelization of the wound followed by cell remodeling.

The above-described embodiments are intended to be examples only. Alterations, modifications and variations can be effected to the particular embodiments by those of skill in the art. The scope of the claims should not be limited by the particular embodiments set forth herein, but should be construed in a manner consistent with the specification as a whole.

What is claimed is:

1. A wound dressing comprising: a pressure dressing, a moisture control core, and a substrate comprising an antimicrobial agent, the substrate in fluid communication with at least a portion said moisture control core, wherein said moisture control core is adapted to supply moisture to a wound.

2. A wound dressing comprising: a pressure dressing, and a moisture control core, wherein said moisture control core is adapted to supply moisture to a wound.

3. The wound dressing of claim 2, further comprising a substrate comprising an antimicrobial agent, the substrate in fluid communication with at least a portion said moisture control core.

4. A wound dressing comprising: a moisture control core, and a substrate comprising an antimicrobial agent, the substrate in fluid communication with at least a portion said moisture control core, wherein said moisture control core is adapted to supply moisture to a wound.

5. The wound dressing of claim 4, further comprising a pressure dressing.

6. A wound dressing comprising: an elastomeric pressure dressing comprising indicia on an outer surface of said pressure dressing; a moisture control core; and a substrate comprising an antimicrobial agent, the substrate in fluid communication with at least a portion said moisture control core, wherein said moisture control core is adapted to supply moisture to a wound.

7. The wound dressing of claim 1, 3, 4, 5, or 6 wherein said antimicrobial agent is a noble metal or metallic ion with antimicrobial properties.

8. The wound dressing of claim 1, 3, 4, 5, 6 or 7, wherein said antimicrobial agent is Ag, Au, Pt, Pd, Ir, Cu, Sn, Sb, Bi, or Zn.

9. The wound dressing of claim 7 or 8, wherein said noble metal is in a concentration of about 1 ppm to about 3025 ppm.

10. The wound dressing of claim 7, 8, or 9, wherein said noble metal is in a concentration of about 50 ppm to about 200 ppm.

11. The wound dressing of claim 1, 3, or 5, wherein said antimicrobial agent comprises silver nanoparticles, elemental silver, zero valent silver, multivalent silver ions carried by zirconium phosphate (ZP-Ag), silver containing compounds such as silver sulfadiazine, or related compounds.

12. The wound dressing of any one of claims 1 to 11, said substrate further comprising a therapeutic agent.

13. The wound dressing of claim 12, wherein said therapeutic agent is one or more of an antibiotic, an anti-viral agent, an anti-protozoal agent, an anti-parasitic agent, or an anti-inflammatory agent.

14. The wound dressing of claim 13, wherein said antibiotic is a β-lactam antibiotics, macrolides, monobactams, rifamycins, tetracyclines, chloramphenicol, clindamycin, lincomycin, fusidic acid, novobiocin, fosfomycin, fusidate sodium, capreomycin, colistimethate, gramicidin, minocycline, doxycycline, bacitracin, erythromycin, nalidixic acid, vancomycin, or trimethoprim.

15. The wound dressing of claim 14, wherein said β-lactam antibiotics is ampicillin, aziocillin, aztreonam, carbenicillin, cefoperazone, ceftriaxone, cephaloridine, cephalothin, cloxacillin, moxalactam, penicillin G, piperacillin, ticarcillin, or any combination thereof.

16. The wound dressing of any one of claims 12 to 15, wherein said anti-inflammatory agent is a steroidal anti-inflammatory or non-steroidal anti-inflammatory.

17. The wound dressing of any one of claims 1 to 16, said substrate further comprising an anesthetic agent.

18. The wound dressing of any one of claims 1 to 17, wherein said substrate comprises Acticoat®.

19. The wound dressing of any one of claims 1 to 18, wherein said moisture control core comprises cotton, rayon, rayon/polyester, cellulose, or cellulose derivatives.

20. The wound dressing of any one of claims 1 to 19, wherein said moisture control core comprises surgical gauze.

21. The wound dressing of any one of claims 1 to 20, wherein said moisture control core has a width of about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, about 20 mm, about 21 mm, about 22 mm, about 23 mm, about 24, or about 25 mm.

22. The wound dressing of any one of claims 1 to 21, wherein said pressure dressing comprises gauze, adhesive tape, bandages, steri-strips, adhesive bandages and pads, a chest binder or front-closing brassiere.

23. The wound dressing of any one of claims 1 to 21, wherein said pressure dressing comprises steri-strips, wherein said moisture control core comprises surgical gauze, and said substrate comprises Acticoat®.

24. The wound dressing of any one of claims 1 to 23, wherein said moisture control core is wetted with a fluid.

25. The wound dressing of claim 24, wherein said fluid comprises water.

26. A method of treating a wound subject, comprising: applying a wound dressing according to any one of claims 1 to 25 to the wound on the subject, wherein said wound dressing is maintained on the wound for a duration of at least about three days, and applied at a pressure of about 10 mmHg to about 100 mmHg.

27. The method of claim 26, wherein said duration is about 3 to about 14 days, about 3 to about 21 days, about 3 to about 28 days, about 3 to about 35 days, about 3 to about 42 days, about 3 to about 49 days, about 3 to about 56 days, or greater than about 56 days.

28. The method of claim 26 or 27, wherein said pressure is about 10 mmHg, about 20 mmHg, about 30 mmHg, about 40 mmHg, about 50 mmHg, about 60 mmHg, about 70 mmHg, about 80 mmHg, about 90 mmHg, or about 100 mmHg.

29. The method of any one of claims 26 to 28, where said moisture control core maintains an environment with about 80% to about 100% relative humidity at the wound surface.

30. A use of a wound dressing according to any one of claims 1 to 25, for treating a wound in a subject, said wound dressing is adapted for application to said wound on said subject for a duration of at least about three days, at a pressure of about 10 mmHg to about 100 mmHg.

31. The use of claim 30, wherein said duration is about 3 to about 14 days, about 3 to about 21 days, about 3 to about 28 days, about 3 to about 35 days, about 3 to about 42 days, about 3 to about 49 days, about 3 to about 56 days, or greater than about 56 days.

32. The use of claim 30 or 31, wherein said pressure is about 10 mmHg, about 20 mmHg, about 30 mmHg, about 40 mmHg, about 50 mmHg, about 60 mmHg, about 70 mmHg, about 80 mmHg, about 90 mmHg, or about 100 mmHg.

33. The use of any one of claims 30 to 32, where said moisture control core maintains an environment with about 80% to about 100% relative humidity at the wound surface.

34. A wound dressing kit, comprising: a pressure dressing, a moisture control core, and a substrate comprising an antimicrobial agent, the substrate adapted for fluid communication with at least a portion said moisture control core, wherein said moisture control core is adapted to supply moisture to a wound.

35. A wound dressing kit comprising: a pressure dressing, and a moisture control core, wherein said moisture control core is adapted to supply moisture to a wound.

36. A wound dressing kit, comprising: an elastomeric pressure dressing comprising indicia on an outer surface of said pressure dressing; a moisture control core; and a substrate comprising an antimicrobial agent; the substrate adapted for fluid communication with at least a portion said moisture control core; wherein said moisture control core is adapted to supply moisture to a wound.

37. The wound dressing kit of claim 35 or 36, further comprising a substrate comprising an antimicrobial agent, the substrate in fluid communication with at least a portion said moisture control core.

38. A wound dressing kit comprising: a moisture control core, and a substrate comprising an antimicrobial agent, the substrate in fluid communication with at least a portion said moisture control core.

39. The wound dressing kit of claim 38, further comprising a pressure dressing.

40. The wound dressing kit of claim 34, 36, 37, or 38, wherein said antimicrobial agent is a noble metal or metallic ion with antimicrobial properties.

41. The kit of claim 34, 36, 37 or 38, wherein said antimicrobial agent is a noble metal or metallic ion with antimicrobial properties.

42. The kit of claim 34, 36, 37, or 38, wherein said antimicrobial agent is Ag, Au, Pt, Pd, Ir, Cu, Sn, Sb, Bi, or Zn.

43. The kit of claim 34, 36, 37 or 38, wherein said noble metal is in a concentration of about 1 ppm to about 100 ppm.

44. The kit of any one of claims 38 to 43, wherein said noble metal is in a concentration of about 50 ppm to about 80 ppm.

45. The kit of any one of claims 38 to 44, wherein said antimicrobial agent comprises silver nanoparticles, elemental silver, zero valent silver, multivalent silver ions carried by zirconium phosphate (ZP-Ag), silver containing compounds such as silver sulfadiazine, or related compounds.

46. The kit of any one of claims 38 to 45, said substrate further comprising a therapeutic agent.

47. The kit of claim 46, wherein said therapeutic agent is one or more of an antibiotic, an anti-viral agent, an anti-protozoal agent, an anti-parasitic agent, or an anti-inflammatory agent.

48. The kit of claim 47, wherein said antibiotic is a β-lactam antibiotics, macrolides, monobactams, rifamycins, tetracyclines, chloramphenicol, clindamycin, lincomycin, fusidic acid, novobiocin, fosfomycin, fusidate sodium, capreomycin, colistimethate, gramicidin, minocycline, doxycycline, bacitracin, erythromycin, nalidixic acid, vancomycin, or trimethoprim.

49. The kit of claim 48, wherein said β-lactam antibiotics is ampicillin, aziocillin, aztreonam, carbenicillin, cefoperazone, ceftriaxone, cephaloridine, cephalothin, cloxacillin, moxalactam, penicillin G, piperacillin, ticarcillin, or any combination thereof.

50. The kit of any one of claims 47 to 49, wherein said anti-inflammatory agent is a steroidal anti-inflammatory or non-steroidal anti-inflammatory.

51. The kit of any one of claims 35 to 50, wherein said substrate comprises Acticoat®.

52. The kit of any one of claims 35 to 51, wherein said moisture control core comprises cotton, rayon, rayon/polyester, cellulose, or cellulose derivatives.

53. The kit of any one of claims 35 to 52, wherein said moisture control core comprises surgical gauze.

54. The kit of any one of claims 35 to 53, wherein said moisture control core has a width of about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, about 20 mm, about 21 mm, about 22 mm, about 23 mm, about 24, or about 25 mm.

55. The kit of any one of claims 35, 36, 37, and 39 to 54, wherein said pressure dressing comprises gauze, adhesive tape, bandages, steri-strips, or adhesive bandages and pads.

56. The kit of any one of claims 35, 36, 37, and 39 to 54, wherein said pressure dressing comprises steri-strips, wherein said moisture control core comprises surgical gauze, and said substrate comprises Acticoat®.

57. The kit of any one of claims 35 to 56, further comprising a fluid.

58. The kit of claim 57, wherein said fluid comprises water.

59. A method as described herein.

60. A use as described herein.

61. A wound dressing as described herein.

62. A kit as described herein.
